(19) United States
(12) Patent Application Publication
(10) Pub. No.: US 2022/0145397 A1
(43) Pub. Date: May 12, 2022

(54) CANCER DIAGNOSIS AND PROGNOSIS

(71) Applicant: OncoLab Diagnostics GmbH, Wiener Neustadt (AT)

(72) Inventors: Robert ZEILLINGER, Wiener Neustadt (AT); Eva OBERMAYR, Vienna (AT); Gerhard HAMILTON, Vienna (AT)

(21) Appl. No.: 17/437,381

(22) PCT Filed: Mar. 2, 2020
(86) PCT No.: PCT/EP2020/055437

§ 371 Date: Sep. 8, 2021

(30) Foreign Application Priority Data
Mar. 8, 2019 (EP) 19161742.2

(51) Int. Cl. C12Q 1/6886

(52) U.S. Cl. CPC C12Q 1/6886; C12Q 2600/158; C12Q 2600/118; C12Q 2600/112

(57) ABSTRACT

A method of identifying an SCLC patient at risk of disease progression comprising determining the expression of at least one marker selected from a neuroendocrine marker and/or DLLS in a blood sample or blood sample fraction of said patient, wherein said at least one marker is indicative of the patients prognosis.

### FIELD OF THE INVENTION

The invention refers to molecular characterization of blood samples in cancer patients for diagnosis and to assess the patients' prognosis.

### BACKGROUND OF THE INVENTION

Lung cancer is the most common cancer worldwide. The estimated number of 2.1 million new cases corresponds to 11.6% of all diagnosed cancer in 218. There are two major types of lung cancer: non-small cell lung cancer (NSCLC) account for 85% of all lung cancers, whereas small-cell lung cancer (SCLC), a highly aggressive neuroendocrine tumor, is diagnosed in just 15% of the cases. Most patients present with metastatic disease with tumor cells circulating in the blood. A reliable differential diagnosis of SCLC vs NSCLC is an important point due to the major differences in the therapeutic approach of these subtypes.

Stovold et al. (British Journal of Cancer (2013) 108, 1704-1711) describes a neuroendocrine serum tumor marker pro-opiomelanocortin (POMC) in an SCLC tumor model.

### SUMMARY OF THE INVENTION

It is the objective to provide a fast, reliable method of determining SCLC in a patient and to assess the patient's prognosis. A specific objective is to provide a reliable diagnostic test for diagnosis or prognosis of SCLC disease, in particular for the purpose of specific therapy guidance, stratification, monitoring and/or control in a patient suffering from SCLC disease and receiving treatment with one or more therapeutics.

The objective is solved by the subject of the claims and as further described herein.

The invention provides for a method of identifying an SCLC patient at risk of disease progression comprising determining the expression of at least one marker selected from a neuroendocrine marker and/or DLL3 in a blood sample or blood sample fraction of said patient, wherein said at least one marker is indicative of the patient's prognosis.

Specifically, said at least one neuroendocrine marker is CgA and/or SYP.

Specifically, said blood sample fraction is a blood fraction enriched in circulating tumor cells (CTCs).

Specifically, CTCs can be isolated from said blood sample or fraction, to further determine marker expression of said CTCs.

Specifically, expression is determined by measuring the level of an expression product of said at least one marker. Specifically, overexpression of said at least one marker is indicative of a high risk of disease progression or progressive SCLC disease.

Specifically, expression is determined by comparing the level of expression product in said sample to a predetermined reference value, wherein an elevated level is indicative of the risk of SCLC, an advanced SCLC disease condition and/or disease progression.

The invention further provides for a method of predicting a cancer disease condition, such as an SCLC disease condition, in a subject not suffering from said disease condition, comprising:

a. determining the level of at least one marker as described herein, in a sample of said subject, and

b. comparing said level to a predetermined reference value of said at least one marker,

wherein an elevated level is indicative of the risk of an advanced cancer disease condition and/or disease progression.

Specifically, the method described herein is applied to predict a cancer disease condition before treatment initiation in a patient to reduce the risk of developing such disease condition, or to prevent such disease condition. Specifically, said cancer disease condition is an SCLC disease condition.

Specifically, the subject can be a patient newly diagnosed and histologically documented with cancer, such as SCLC, before starting treatment, to decide on the appropriateness of immunotherapy (e.g. DLL3 targeting therapy, if a DLL3 marker was overexpressed) or chemotherapy e.g., as a first-line therapy, and/or the number of chemotherapy cycles, radiotherapy, prophylactic cranial irradiation (PCI), or the like.

Specifically, the subject can be an early stage cancer patient before the initiation of chemotherapy to reduce the risk of acquiring a more advanced stage of disease, or disease progression e.g., by a second-line chemotherapy.

When expressing said at least one marker described herein is determined positive, early stage cancer patients can be treated as if the cancer has already advanced to a later stage, or to progressive cancer, according to applicable clinical guidelines.

The markers described herein are also referred to as “biomarkers”, and a collection (e.g., a combination or array) of markers is also referred to as “panel”. The markers described herein are particularly characterized by their coding nucleotide sequence, or the encoded amino acid sequence. Exemplary protein sequences to the recited markers are provided herein. Protein sequences may or may not comprise an N-terminal secretion signal sequence. Such signal sequence is typically not meant to be part of the marker sequence. It is understood that a marker is specifically characterized by a respective marker coding sequence (or gene), or sequences encoded by a respective marker gene, including naturally-occurring isomers.

The invention further provides for a method of diagnosing SCLC in a lung cancer patient comprising determining the expression of at least one marker selected from a neuroendocrine marker and/or DLL3 in a fraction of a blood sample of said patient and SCLC patient, which blood fraction is enriched in circulating tumor cells (CTCs), wherein said at least one marker is indicative of SCLC.

The invention further provides for a method of monitoring treatment of an SCLC patient with a therapy, comprising determining the expression of at least one marker selected from a neuroendocrine marker and/or DLL3 in a fraction of a blood sample of said patient which blood fraction is enriched in circulating tumor cells (CTCs), wherein said at least one marker is indicative of the patient's response to said therapy.

Specifically, blood samples are drawn at least twice, at two different consecutive time points during the therapy.

Specifically, the patient is treated with a therapy used for treating advanced SCLC disease, e.g., chemotherapy and/or immunotherapy and/or radiotherapy, in particular chemoradiation therapy.

Specifically, one or more of said markers described herein can be used for monitoring or surveillance. For example, the biomarker can be used for surveillance purposes supporting the physician predict or monitor the progression of disease.

Specifically, the method described herein is used for therapy guidance, stratification, monitoring and/or control, in particular employing an appropriate immunotherapy or chemotherapy to mitigate the risk. To this end, the method described herein specifically further comprises applying, maintaining, reducing, elevating or not applying a therapy based on whether the subject is at said risk of disease or disease progression.

The invention further provides for a method of monitoring a cancer disease condition, such as an SCLC disease condition, in a subject not suffering from said disease condition, comprising:

a. determining the level of one or more markers as described herein, in a sample of a subject at a first time point, and

b. determining the level of said one or more markers as described herein, in a sample of the same subject at a later second time point,

wherein an increase in the level between the first and second time points is indicative of the onset of SCLC disease, or the onset of advanced SCLC disease and/or disease progression.

Specifically, the risk of an SCLC disease condition (or the onset of such disease condition) is indicated if the biomarker level is higher than expected for the subject. For example, the onset of an SCLC disease condition is indicated or predicted, if the respective marker level in the sample is higher than a predetermined reference level normally found in the patient population that does not suffer from said an SCLC disease condition, or that does not develop such SCLC disease condition.

The invention further provides for a method of monitoring the effectiveness of at least one treatment applied to a subject for treating a cancer disease condition, such as an SCLC disease condition, comprising:

a. determining the level of one or more markers as described herein, in a sample of a subject at a first time point; and

b. determining the level of said one or more markers as described herein, in a sample of the same subject at a later second time point, and

wherein a decrease in the level between the first and second time points is indicative of treatment success, or indicative of a lower risk of any subsequent disease progression.

To provide for comparability or the results, samples drawn at different time points, such as the first and second time points referred to herein, are particularly of the same type, and treated the same way.

As used herein, “diagnosis” relates to the recognition and (early) detection of a clinical condition of a subject linked to a cancer disease or a cancer disease condition. Also the assessment of the stage of cancer disease may be encompassed by the term “diagnosis”. “Prognosis” relates to the prediction of an outcome or a specific risk for a subject. This may also include an estimation of the chance of recovery or the chance of a disease progression for said subject.

The methods described herein may also be used for monitoring, therapy monitoring, therapy guidance and/or therapy control. “Monitoring” relates to keeping track of subject such as a cancer patient e.g., suffering from lung cancer or SCLC, and potentially occurring disease progression or complications, e.g. to analyze the progression of the healing process or the influence of a particular treatment or therapy on the health state of the patient. Specifically, the patient is monitored by determining and comparing the expression level of said at least one marker at different timepoints.

The term “indicate” as used herein e.g., in the context of indicating an event, such as a disease condition, the risk of a disease condition, disease progression, or treatment success is herein understood as a measure of risk and/or likelihood. Preferably, the “indication” of the presence or absence of an event is intended as a risk assessment, and is typically not to be construed in a limiting way as to point definitively to the absolute presence or absence of said event.

The term “cancer disease condition” as used herein is specifically understood to refer to cancer as a disease or certain stage of such disease, such as an early, progressive, advanced or late stage of cancer. An advanced cancer disease condition is herein specifically understood as stage III or IV of disease.

The term “SCLC disease condition” as used herein is specifically understood to refer to SCLC cancer as a disease or certain stage of such disease, such as an early, progressive, advanced or late stage of SCLC. An advanced SCLC disease condition is herein specifically understood as stage III or IV of disease.

Specifically, the subject (or patient) is a human being. Yet, according to a specific aspect, the subject can be a non-human animal e.g., an animal used for experimental methods of disease treatment, such as used in animal models.

Specifically, the patient is a lung cancer patient where SCLC has not been diagnosed before. According to a specific aspect, the patient is a lung cancer patient, where the diagnosis of NSCLC or SCLC has not yet been confirmed by histopathology.

Specifically, the patient is a NSCLC patient who develops into a SCLC patient.

Specifically, the patient is an SCLC patient which has a high risk of progressive disease or developing an advanced stage of SCLC, e.g. despite of standard therapy.

Specifically, expression is determined by measuring RNA or protein expression.

Specifically, expression of said at least one marker is determined by determining expression products, such as those encoded by a marker gene, or those of the respective complementary sequence, including e.g., polypeptides, proteins, transcripts, like mRNA, or micro-RNA. The term “mRNA” as used herein shall refer to any of pre-mRNA transcript(s), transcript processing intermediates, mature mRNA(s) ready for translation and transcripts of the gene or genes, or nucleic acids derived from the mRNA transcript(s). A nucleic acid derived from an mRNA transcript is herein understood to refer to a nucleic acid for whose synthesis the mRNA transcript or a subsequence thereof has ultimately served as a template. A cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “expression” as used herein, is meant to encompass at least one step selected from the group consisting of DNA transcription into mRNA, mRNA processing, mRNA maturation, mRNA export, translation, protein folding and/or protein transport.

Specifically, an expression level is determined quantitatively or semi-quantitatively.

Specifically the nucleic acid and/or protein expression is determined, either qualitatively or quantitatively. The specific marker described herein is preferably determined by testing for the respective expression products, such as expressed polypeptides (or proteins) and/or polynucleotides (or nucleic acid molecules), like mRNA, indicative of marker expression.

Specifically, the amount or level of any such expression product is determined by suitable analytical methods and means.

Specifically, expression is determined in a blood sample e.g. upon fractionation and/or enrichment of CTCs, or in a CTC comprising fraction of a blood sample. Specifically, it is determined whether CTCs in a sample are positive in expressing any one or more of said markers described herein.

Specifically, expression is determined by measuring an expression product by RT-PCR or an immunoassay.

Specifically, the RNA-expression is measured by any of a nucleic amplification method, hybridizing or sequencing method. The expression is optionally employing amplification methods, among them signal or nucleic acid amplification methods, such as RT-qPCR. Specifically, an expression product being a marker nucleic acid is measured by a real-time reverse transcriptase PCR assay or a nucleic acid microarray assay.

Specifically, the RNA-expression is determined by using a template sequence or comparing to such template sequence, corresponding to the nucleotide sequence encoding the respective amino acid sequence, identified as SEQ ID NO:1, 2, or 3, and optionally SEQ ID NO:4 and/or 5.

Specifically, the RNA-expression is determined by measuring the mRNA expression. According to a specific example, the mRNA of the individual genes encoding the respective SEQ ID NO:1, 2, or 3, and optionally SEQ ID NO:4 and/or 5, in the sample, as determined either quantitatively or qualitatively, e.g. the differential expression, such as an increased or decreased expression, is determined.

Specifically, expression products can be determined applying an analytical method selected from the group consisting of mass spectrometry (MS), luminescence immunoassay (LIA), radioimmunoassay (RIA), chemiluminescence- and fluorescence-immunoassays (e.g. immunofluorescence staining), enzyme immunoassay (EIA), Enzyme-linked immunoassays (ELISA), luminescence-based bead arrays, magnetic beads based arrays, protein microarray assays, rapid test formats e.g., immunochromatographic strip tests, and automated systems/analyzers.

According to a specific aspect, the level of an expression product being a marker protein is determined using an immunoassay, such as an immunohistochemical assay, an immunoblotting assay, or a flow cytometry assay.

Specific analytical methods may employ a ligand which is an immunoagent, such as an immunoagent for use in an immunoassay.

Specifically, the immunoassay is selected from the group consisting of an enzymatic immunoassay, such as an ELISA (e.g. a sandwich ELISA), lateral flow immunochromatographic assay, fluorescent immunoassay, radioimmunoassay, and magnetic immunoassay.

Specifically, the analytical method may employ detection of a label, preferably an enzymatic label, a fluorescent label, or a radioisotope label.

According to a specific aspect, the method described herein can be combined with any other diagnostic method for determining lung cancer, or SCLC, specifically another diagnostic method of imaging and/or histopathology.

Specifically the expression is determined qualitatively and/or quantitatively, e.g. by Northern blot or other hybridization based methods, RT-qPCR or other nucleic amplification methods, microarrays, sequencing methods, or ligand binding assays. Specific methods of determining the expression of genes or non-coding sequences associated therewith, including any of corresponding RNA, such as mRNA, or cDNA specimen, or any other expression products are preferred.

Specifically, the blood sample is a sample of circulating peripheral blood.

Specifically, the blood fraction enriched in CTCs is a CTC fraction obtained by separating a non-cellular fraction, e.g. serum, from a blood sample.

According to a specific aspect, the sample is obtained from a blood fraction enriched in white blood cells, including lymphocytes and optionally granulocytes. Preferably the PBC fraction is used, wherein as in white blood cells a majority of granulocytes is contained. An exemplary blood fraction is obtained by density fractionation, such as buoyant one or more-step gradient procedures to obtain a fraction with a high density, which contains the lymphocytes and granulocytes, e.g. as obtained by a sample preparation method as described in Brandt et a. (Clinical Chemistry 42(11), 1881-1882 (1996), or alternative methods to obtain a PBC fraction, which is a white blood cell fraction containing mainly lymphocytes. Besides, other white blood cells which are not lymphocytes may be contained.

Such PBC fraction is preferred that is obtained upon increasing the content of potentially present epithelial cells, including circulating tumor cells, which includes (partial or quantitative) enrichment or isolation of epithelial cells.

The preferred method employs a sample, which is obtained from a fraction containing circulating tumor cells.

Specifically, said at least one marker is indicative of the patient's prognosis, if higher than a reference value. Specifically, such reference value is at least the detection limit of the method. In specific embodiments, the marker is positive, if the respective expression product, e.g., RNA, is detected, and negative, if the respective expression product, e.g., RNA, is below the detection limit.

Specifically, said at least one marker is indicative of SCLC, if higher than a reference value. Specifically, such reference value is at least the detection limit of the method.

Specifically, said at least one marker is indicative of the patient's response to SCLC therapy, if higher than a reference value. Specifically, such reference value is at least the detection limit of the method.

Specifically, the level of expression, in particular RNA-expression such as mRNA, of a marker described herein is determined.

The term “level” is herein understood as the absolute or relative amount or concentration of a marker, a presence or absence of the marker, a range of amount or concentration of the marker, a minimum and/or maximum amount or concentration of the marker, a mean amount or concentration of the marker, and/or a median amount or concentration of the marker. Specific levels are provided as a value of the amount representing the concentration, and specifically expressed as weight/volume; w/v, or expressed as fold change of the marker in the sample.

The term “level” in relation to a marker described herein is understood as the level of such marker in a sample which is greater than a reference or standard level, such reference may be a cutoff value of the marker. Positive predictive values or negative predictive values may be used as a reference.

An elevated level is herein understood as a significantly higher level than the reference. The term “significant” with respect to the overexpression of the marker as used herein shall refer to at least a two-fold higher amount of the standard deviation, preferably at least a three-fold difference. In a preferred quantitative determination method, the expression of the marker is normalized to the median expression of one or more reference genes, e.g. used as internal control.

With respect to a specific reference value, such as derived from a standard, training data or threshold, a significant elevated or increased amount is understood to refer to an at least 1.5-fold higher amount, preferably at least 2- or 3-fold difference.

The reference level may be a value resulting from calibrating the method against samples of patients with a known disease or disease condition, or risk of such disease or disease condition, or disease progression. Specifically, the reference level indicating a high risk of cancer disease, in particular SCLC or advanced SCLC disease, or disease progression is higher than such reference level indicating a lower risk.

Specifically, the methods described herein may be quantitative or semi-quantitative methods such as determining whether the biomarker level in the sample is above a reference or threshold level.

Specifically, the reference level is a threshold, also understood as a cut-off value indicating a marker concentration for the respective risk or the severity of disease. The respective concentrations that determine the threshold values depend on multiple parameters such as the time point of sample isolation and the assay or detection used for determining the biomarker level in said sample.

A threshold level may distinguish between healthy subjects and diseased subjects, and/or between subjects at low risk of developing disease or disease progression and those subjects who are at a higher risk of developing disease or disease progression. Low risk subjects can be e.g., healthy subjects, or those with an early stage of disease and/or stable disease. High risk subjects can be e.g., at the onset of disease, or already suffering from disease.

As used herein, the term “cutoff value” particularly refers to a threshold value which distinguishes subjects who are suffering from a disease or disease condition from a population of patients and/or subjects who are not suffering from the disease or disease condition, and in particular distinguishes subjects who are at high risk of disease or disease progression from a population of patients and/or subjects who are not at such high risk of disease or disease progression.

The relevant reference levels can be determined by well-known methods e.g. based on extensive data which can be routinely obtained by comparing samples from diseased patients with healthy subjects or subjects not suffering from such disease. Such references are understood as population averages levels, for example mean marker population values, whereby patients that are diagnosed as cancer patient, such as lung cancer or SCLC patients, may be compared to a control population, wherein the control group preferably comprises more than 10, 20, 30, 40, 50 or more subjects.

Appropriate normal reference levels of the marker may be determined by measuring levels of such marker in one or more appropriate subjects, and such reference levels may be tailored to specific populations of subjects (e.g., a reference level may be age-matched or gender-matched so that comparisons may be made between marker levels in samples from subjects of a certain age or gender and reference levels for a sepsis condition state, phenotype, or lack thereof in a certain age or gender group).

According to a specific aspect, a software system can be employed, in which a machine learning algorithm is evident, preferably to identify subjects suffering from a cancer disease condition, or at risk for a cancer disease condition, using data from electronic health records (EHRs). A machine learning approach can be trained on a random forest classifier using EHR data (such as labs, biomarker expression, vitals, and demographics) from patients. Machine learning is a type of artificial intelligence that provides computers with the ability to learn complex patterns in data without being explicitly programmed, unlike simpler rule-based systems.

The invention further provides for a set of reagents (a kit of parts) to determine the expression of any of the markers further described herein. The term “kit” is specifically understood as “diagnostic kit” and refers to a kit or set of parts, which in combination or mixture can be used to carry out the measurement/detection of one or more analytes or markers.

Specifically, the kit described herein may contain at least a detection molecule and/or a binder (e.g. a primer, probe or an immunoagent), wherein the detection molecule and/or the binder specifically recognizes the marker or the respective analyte, or a reaction product of such marker or analyte. In addition, various reagents or tools may be included in the kit. The diagnostic kit preferably comprises all essential components to determine the amount of the marker in the biological sample, optionally without common or unspecific substances or components, such as water, buffer or excipients that may be conveniently added when performing the analysis. The diagnostic kit may comprise any useful reagents for carrying out the subject methods, including substrates or solid surfaces, such as microbeads or planar arrays or wells, reagents for marker isolation, detection molecules directed to specific targets, detectable labels, solvents, buffers, linkers, various assay components, blockers, and the like.

A kit (herein also referred to as “set”) may also include instructions for use in a diagnostic method. Such instructions can be, for example, provided on a device included in the kit, e.g. tools or a device to prepare a biological sample for diagnostic purposes, such as separating a cell and/or protein containing fraction before determining a marker. The kit may conveniently be provided in the storage stable form, such as a commercial kit with a shelf-life of at least 6 months. The storage stable kit can be stored preferably at least 6 months, more preferably at least 1 or 2 years. It may be composed of dry (e.g. lyophilized) components, and/or include preservatives.

The preferred diagnostic kit is provided as a packaged or prepackaged unit, e.g. wherein the components are contained in only one package, which facilitates routine experiments. Such package may include the reagents necessary for one or more tests, e.g. suitable to perform the tests of a series of biological samples. The kit may further suitably contain a marker preparation as a standard or reference control, which may be the specific marker described herein which comprises a label.

According to a specific embodiment, the set further comprises means to prepare a CTC fraction of a blood sample. Such means include buffer or other auxiliary reagents or tools to enrich and/or fractionate white blood cells, cell lysis reagents, internal controls, negative controls, etc.

The preferred set further contains reagents for determining the expression of at least one of the markers further described herein, e.g., primers, optionally together with one or more probes, to perform an RT-qPCR analysis of a marker, optionally together with further tools to perform such analysis.

The set may comprise one or more primers or probes which correspond to or specifically hybridise with gene transcription products of the individual markers.

According to a further specific aspect, a preferred set contains or one or more ligands specifically recognizing a marker as a binding partner, and/or reagents specifically recognizing the product of binding the ligand to such marker.

The term “specifically recognizing” as used herein (also referred to as “specific binding”) is meant that the binding partner or ligand reacts or associates more frequently, more rapidly, with greater duration and/or with greater affinity with a particular target biomarker than it does with alternative biomarkers.

Marker specific ligands are substances which can bind to or detect at least one of the markers for a detection method described herein and are in particular marker nucleotide sequence detecting tools or marker protein specific antibodies, including antigen-binding antibody fragments, such as Fab, F(ab), F(ab)′, Fv, scFv, or single chain antibodies. Marker specific substances can also be selected from marker nucleotide sequence specific oligonucleotides, which specifically bind to a portion of the marker sequences, e.g. mRNA or cDNA, or are complementary to such a portion in the sense or complementary anti-sense orientation, like a cDNA complementary strand. The preferred ligands may be attached to solid surfaces, including beads, to catch and separate the marker, or CTC expressing the marker in the sample.

According to specific embodiments, a ligand can be labelled. Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Specific ligands are immunoagents, such as those particularly used in an analytical method.

The term “immunoagent” as used herein is understood as a target-specific molecule that contains a target binding site that specifically binds or immunoreacts with said target, such as a target antigen. Preferred immunoagents are antibodies or antigen-binding fragments thereof e.g., monoclonal or polyclonal antibodies or respective antibody fragments. Particularly, antibodies that are specifically binding to the biomarker are employed in an immunoassay for determining the biomarker.

Specific immunoagents may be capture molecules or molecular scaffolds, which are understood as molecules that may be used to bind target molecules or molecules of interest, i.e. analytes such as the marker described herein, from a sample. Such molecules are shaped adequately, both spatially and in terms of surface features, such as surface charge, hydrophobicity, hydrophilicity, presence or absence of Lewis donors and/or acceptors, to specifically bind the target molecule. Hereby, the binding may, for instance, be mediated by ionic, van-der-Waals, pi-pi, sigma-pi, hydrophobic or hydrogen bond interactions or a combination of two or more of the aforementioned interactions or covalent interactions between the capture molecules or molecular scaffold and the target molecule. Capture molecules or molecular scaffolds may for instance be selected from the group consisting of a nucleic acid molecule, a carbohydrate molecule, a PNA molecule, a protein, a peptide and a glycoprotein, for example, aptamers, DARpins (Designed Ankyrin Repeat Proteins), Affimers and the like.

Specifically, the immunoagent is considered to be specifically recognizing a target such as a marker described herein, if its affinity towards the target is at least 100-fold or 1000-fold higher than towards other molecules comprised in the sample containing the marker. It is well known in the art how to develop and to select antibodies with a given specificity to recognize a target antigen.

According to a specific aspect, the immunoassay may employ at least one antibody which is labeled, and another antibody that is bound to a solid phase or can be bound selectively to a solid phase. The first antibody and the second antibody can be present dispersed in a liquid reaction mixture, and a first labeling component may be bound to the first antibody, and a second labeling component of said labeling system may be bound to the second antibody so that, after binding of both antibodies to the biomarker to be detected, a measurable signal which permits detection of the resulting sandwich complexes in the measuring solution is generated

According to a specific aspect, the method described herein may be performed as an immunoassay comprising the steps of:

a) contacting the sample with a first antibody or an antigen-binding thereof, specific for a first epitope of the marker, and with second antibody or an antigen-binding fragment thereof, specific for a second epitope of the marker; and

b) detecting the binding of the first and second antibodies or antigen-binding fragments thereof to the marker.

In particular, the first antibody and the second antibody may be present dispersed In a liquid reaction mixture, and wherein a first labelling component is bound to the first antibody, and/or a second labelling component of said labelling system Is bound to the second antibody so that, after binding of both antibodies to at least one marker or fragment thereof, a measurable signal which permits detection of the resulting sandwich complexes in the measuring solution is generated.

In specific cases, the method is carried out as sandwich immunoassay, specifically wherein one the antibodies is immobilized on a solid phase, for example, the walls of coated test tubes, microtiter plates, or magnetic particles, and another antibody comprises a detectable label or means enabling for selective attachment to a label, and which serves the detection of the formed sandwich structures.

The set may further suitably contain a preparation as a standard or reference control. Specifically a negative control or reference control composition may be used. Specifically, a positive reference composition may be used.

In such case, a detection signal may be normalized to a control signal corresponding to the amount of signal produced by the control composition, thereby detecting the presence or level of the RNA determined in the sample. Normalizing the detection signal may comprise subtracting the control signal from the detection signal.

The set may be particularly used in a method described herein.

The determination of high or low levels of the markers described herein has turned out to be highly reliable with respect to determining a cancer disease condition, differentiating said cancer from other types of cancer, or cancer disease progression, such that it enables an appropriate action by a medical professional. Specifically, said cancer is SCLC.

### FIGURES

FIG. 1: Cycle threshold (Ct) values of the respective gene transcripts resulting from qPCR analysis of 76 SCLC and 26 HND blood samples.

FIG. 2: Percentage of qPCR-positive blood samples due the expression of the given markers.

FIG. 3: Kaplan-Meyer overall survival curves.

A: lower curve: CgA (CHGA) positive; upper curve: CgA (CHGA) negative.

B: lower curve: DLL3 positive; upper curve: DLL3 negative.

C: lower curve: SYP positive; upper curve: SYP negative.

A negative impact of DLL3 and CgA on overall survival was observed (median OS 4 vs 9 months, log-rand p=0.035 and p=0.024), but not of EpCAM, and CK19.

FIG. 4: qPCR findings in SCLC patients and healthy donors. Cycle threshold (Ct) values of the respective gene transcripts resulting from qPCR analysis of 65 SCLC and 19 HND blood samples.

FIG. 5: Percentage of qPCR-positive blood samples due the expression of the given markers.

FIG. 6: Kaplan-Meyer overall survival curves.

A: lower curve: DLL3 positive; upper curve: DLL3 negative. p (log-rank)=0.007; mean OS 1 vs. 11 months.

B: lower curve: SYP positive; upper curve: SYP negative. p (log-rank)=0.009; mean OS 4 vs. 11 months.

C: lower curve: CgA (CHGA) positive; upper curve: CgA (CHGA) negative. p (log-rank)=0.025; mean OS 4 vs. 11 months.

FIG. 7: Sequences referred to herein

### DETAILED DESCRIPTION

The markers further described herein are the following:

CgA, CHGA

Gene name: Chromogranin-A

Alternative name: Pituitary secretory protein I

Chromosome location: 14

Uniprot: P10645 (CMGA_HUMAN)

SEQ ID NO:1 (human CgA)

SYP

Gene name: synaptophysin, synaptophysis

Alternative name: Major synaptic vesicle protein p38

Chromosome location: X

Uniprot: P08247 (SYPH_HUMAN)

SEQ ID NO:2 (human SYP)

DLL3

Gene name: Delta-like protein 3; delta-like 3 ligand

Alternative name: Drosophila Delta homolog 3

Chromosome location: 19

Uniprot: Q9NYJ7 (DLL3_HUMAN)

SEQ ID NO:3 (human DLL3)

DLL3 is part of the Wnt signaling pathway, which is a group of signal transduction pathways which begin with proteins that pass signals into a cell through cell surface receptors. Wnt is an acronym in the field of genetics that stands for ‘Wingless/Integrated’. The present invention allowed predicting the response of SCLC to certain treatment, such as with rovalpituzumab tesirine (Rova-T). In cases where DLL3 was not detectable or detectable at only low amounts, patients did not respond to such treatment. Therefore, it can be a valuable marker for monitoring therapy, where therapy or continued therapy with second-line treatments (following a first chemotherapy) is indicated. DLL3 may also be useful for the selection of patients, because CTCs enable a real-time measurement of the DLL3-status right before initiation of the respective treatment.

EpCAM

Protein name: Epithelial cell adhesion molecule

UniProtKB—P16422 (EPCAM_HUMAN)

SEQ ID NO:4 (P16422-1)

Cytokeratin—19 (KRT19, CK-19 or CK19)

Protein name: Keratin, type I cytoskeletal 19

Uniprot: P08727 (K1C19_HUMAN)

SEQ ID NO:5 (P08727-1) The present marker(s) can be used as predictive marker(s), e.g. as a stand-alone diagnostic or in combination with further diagnostic measures, such as the determination of further markers (e.g. in a diagnostic combination kit).

According to certain embodiments, the method described herein additionally comprises determining a level of at least one additional marker in a sample from said patient. Specifically, the additional marker(s) are tumor associated marker(s), such as e.g., an epithelial tumor marker selected from the group consisting of EpCAM, CK19, and for example E-cadherin (ECAD). Specifically, the additional marker(s) are lung cancer markers, in particular SCLC markers.

According to a specific aspect, one or more further neuroendocrine markers may be detected according to the invention, such as Insulinoma-associated protein 1, neural cell adhesion molecule 1 (NCAM1), or enolase 2 (ENO2).

In particular aspects, the methods described herein utilize more than one markers in a multimarker panel, which includes at least one of the specific markers described herein. Specifically, the multimarker panel is a cancer multimarker panel which comprises or consists of those markers of relevance to said cancer, in particular lung cancer or SCLC. Specifically, the cancer multimarker panel described herein may provide for an expression profile associated with said cancer. Such profile may provide a highly sensitive and specific test with both high positive and negative predictive values permitting diagnosis and prediction of the patient's risk of developing disease or disease progression, in particular the risk of developing metastatic disease.

The methods described herein specifically refer to measurement of expression of one, two or three of CgA, SYP, or DLL3, and optionally further measuring expression of EpCAM and/or CK19.

Specifically provided herein is a cancer specific multimarker panel comprising or consisting of one, two or three of the biomarkers CgA, SYP, or DLL3, and optionally further comprising the biomarker EpCAM and/or CK19. A preferred multimarker panel comprises or consists of one, two, three, four or five of CgA, SYP, DLL3, EpCAM or CK19. Specifically, a multimarker panel described herein is provided in a cancer specific, in particular lung cancer or SCLC specific, multimarker panel.

Specifically, the multimarker panel comprises or consists of at least 2, 3, 4, or 5 different markers. Specifically, the multimarker panel is composed of a number of different biomarkers, which is less than any one of 100, 90, 80, 70, 60, 50, 40, 30, 20, or 10, in particular less than 10, 9, 8, 7, or 6.

According to a specific aspect, a diagnostic kit is provided which comprises the multimarker panel described herein and ligands specifically recognizing expression products of each marker of said multimarker panel. Preferably, the kit comprises a ligand being a specific binder for each of the markers. Specifically, the diagnostic kit is provided, which does not comprise any further marker panels and further specific specific ligands, other than said multimarker cancer panel described herein and respective specific ligands.

The same or different types of expression products may be determined when analysing a sample using the multimarker panel described herein. In specific embodiments, each marker of a multimarker panel is determined by a polynucleotide, such as mRNA or transcripts, as expression product indicating said marker expression. In further specific embodiments, at least one marker of a multimarker panel is determined by a polypeptide or protein as expression product indicating said marker expression.

In a particular embodiment, DLL3 and/or EpCAM and/or CK19 expression is determined by the expressed polynucleotide, such as mRNA, or the expressed polypeptide or protein. Specifically, DLL3 expression can be determined by the expressed polypeptide or protein.

In a particular embodiment, CgA and/or SYP expression is determined by the expressed polynucleotide, such as mRNA.

The multimarker panel can be placed on a microarray so that the expression status of each of the markers is assessed side-by-side or simultaneously. In an embodiment, a microarray is provided as a prognostic tool comprising a defined set of marker genes, whose expression is significantly altered in lung cancer and which may be determined by hybridization, by amplification of polynucleotides, and/or sequencing.

Standard therapy of progressive SCLC, in particular following identifying the SCLC patients at risk of disease progression using the method further described herein, employs chemotherapy, optionally combined with immunotherapy and/or radiotherapy, as used in SCLC patients of progressive or recurrent disease. Such standard therapy typically follows a surgical intervention to remove tumor tissue.

SCLC is generally treated with combinations of chemotherapy drugs. The combinations most often used are:

Cisplatin and etoposide

Carboplatin and etoposide

Cisplatin and irinotecan

Carboplatin and irinotecan

Specifically, anti-DLL3 therapy (in particular anti-DLL3 immunotherapy) is indicated, if expression of DLL3 is determined in a blood sample, or a CTC containing fraction thereof.

Doctors give chemo in cycles, with a period of treatment (usually 1 to 3 days) followed by a rest period to allow the patient's body time to recover. Each cycle generally lasts about 3 to 4 weeks, and initial treatment is typically 4 to 6 cycles.

If the cancer progresses during treatment as determined by the method further described herein, other chemotherapeutic drugs may be given, such as those used in recurrent SCLC cases, e.g., Topotecan oder Epirubicin.

Rovalpituzumab tesirine (Rova-T) is currently investigated for use as a second-line treatment for patients with advanced small cell lung cancer (SCLC).

In some cases of SCLC patients identified using the method further described herein, immunotherapy is combined with chemotherapy e.g., using a checkpoint inhibitor targeting PD-1, such as nivolumab (Opdivo) which is a drug that targets PD-1, a protein on T cells (a specific type of immune system cell) that normally helps keep these cells from attacking other cells in the body. By blocking PD-1, this drug boosts the immune response against small cell lung cancer cells. It is generally used in people with advanced small cell lung cancer whose cancer continues to grow after at least two previous systemic treatments including cisplatin or carboplatin.

Depending on the stage of small cell lung cancer (SCLC) and other factors, radiation therapy might be used:
radiation therapy can be given at the same time as chemotherapy to treat the tumor and lymph nodes in the chest. Giving chemo and radiation together is called concurrent chemoradiation. The radiation may be started with the first or second cycle of chemotherapy.
Radiation can also be given after the chemo is finished. This is typically done for patients with progressive disease, or an extensive stage disease.
SCLC often spreads to the brain. Radiation can be given to the brain to help lower the chances of problems from cancer spread there. This is called prophylactic cranial irradiation. This is particularly used to treat patients with progressive disease, or an extensive stage disease.

In particular, those therapeutic measures otherwise used for progressive or advanced SCLC disease, can be indicated as a first line therapy, if the SCLC patients have been identified to have a worse prognosis according to the method further described herein, or if they have been determined to be responsive to such therapy (e.g. by the DLL3 expression in circulating CTCs).

SCLC is herein understood to be progressive, if advancing to the next stage(s). According to the TNM system, the earliest stage is stage 0. The other main stages range from I through IV.

Advanced SCLC is herein understood as SCLC that is characterized by a large tumor size and/or spread to nearby (regional) lymph nodes, and/or spread (metastasis) to other organs of the body, such as the brain, bones, adrenal glands, kidneys, liver, or the other lung.

The histopathological diagnosis of the disease is generally made upon assessing the cell morphology in tissue sections derived from biopsy specimens. So-called “liquid biopsies” are taken by circulating (or peripheral) blood draw and are less invasive for the patients than conventional tissue biopsies. Furthermore, they can be taken at serial timepoints, and thus may represent not only the spatial but also temporal heterogeneity of the tumor in the course of the disease. The present study provides a proof-of-principle that circulating tumor cells (CTCs) are assessed in SCLC “liquid biopsies” and expression of certain marker(s) e.g., detected by a qPCR-based method allows treatment indicated for SCLC patients in particular advanced or progressive disease.

### EXAMPLES

### Example 1

Patients and Methods

76 lung cancer patients with a histopathological confirmed diagnosis of SCLC were included into the study. In 43 cases (56.6%) the blood was taken at primary diagnoses, and in 33 cases (43.4%) at disease progression. In addition, blood samples were taken from 26 healthy normal donors (HND) as control.

All blood samples were processed using the Parsortix™ system (Angle plc., UK) to increase the relative abundance of CTCs in the sample.

After harvesting and lysis of the captured cells, the total RNA was extracted and reverse transcribed into cDNA. Finally, qPCR was performed using TagMan® assays specific for EpCAM, NCAM1, CgA, SYP, DLL3, ENO2, CK19, and CDKN1B.

Results

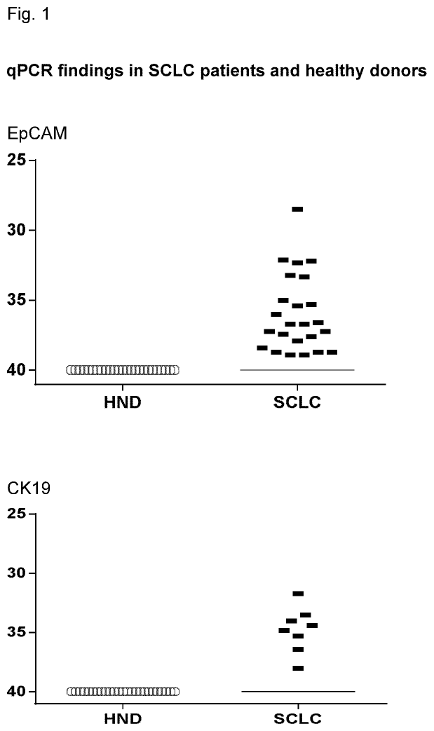

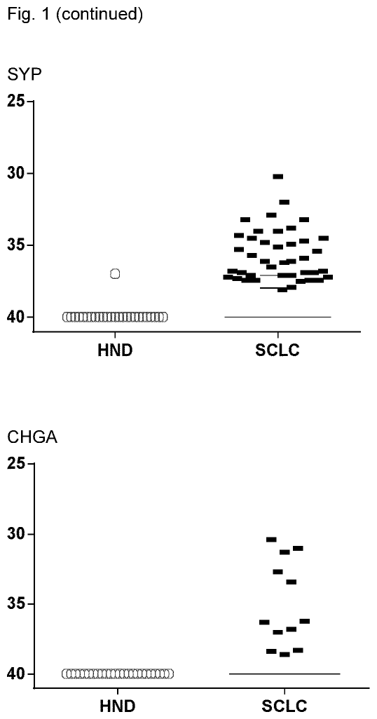

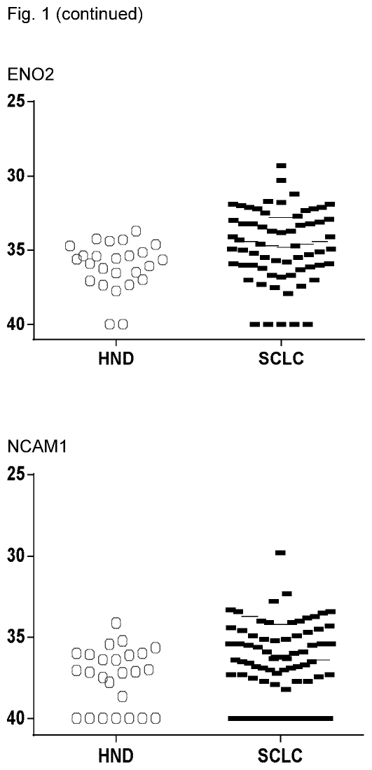

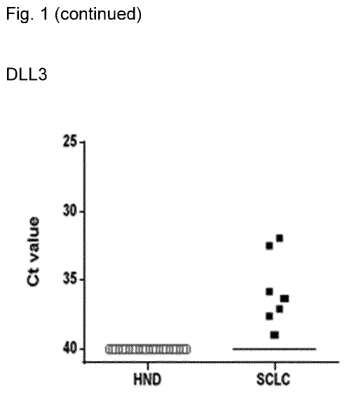

EpCAM, CK19, CgA, and DLL3 transcripts were observed in 31.6%, 10.5%, 14.5%, 9.2% of the 76 SCLC samples, and in none of the 26 HND samples. SYP transcript levels beyond the observed level in the HND samples were observed in 36.8% of the patients. ENO2 and NSAM1 levels were similar in the HND and SCLC group, and thus not considered as appropriate markers for CTC detection (FIG. 1).

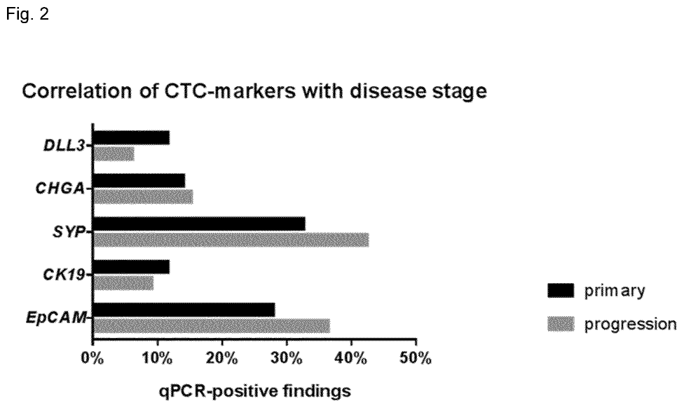

Overall, 38/76 (50%) samples were assigned as CTC-positive due to the expression of at least one marker out of EpCAM, CK19, CgA, DLL3 and SYP. The expression of epithelial markers (EpCAM and CK19) was observed in 25/76 (32.9%) patients, of neuroendocrine markers (CgA, SYP), and DLL3, in 30/76 (39.5) cases. 17/76 (22.3) cases were characterized by the presence of both types of markers. The percentage of positive samples was not associated with the stage of disease when the blood sample was taken (FIG. 2).

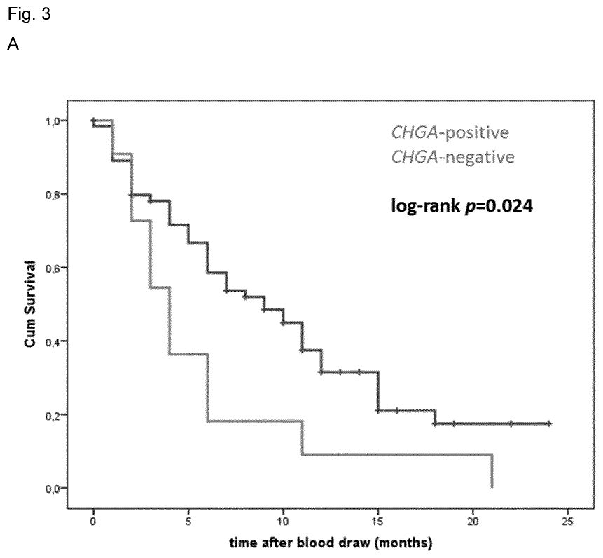

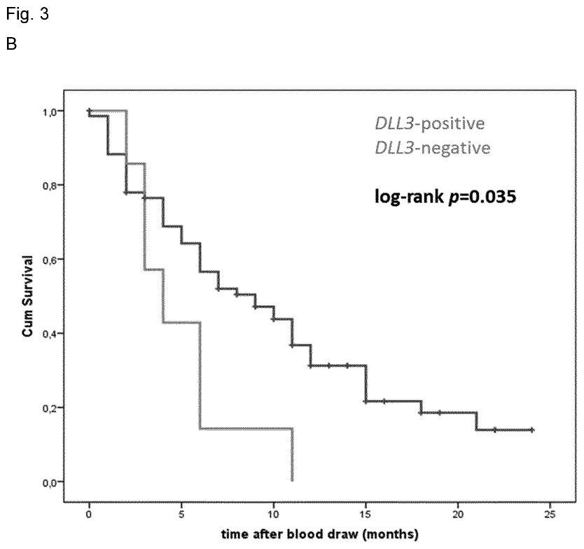

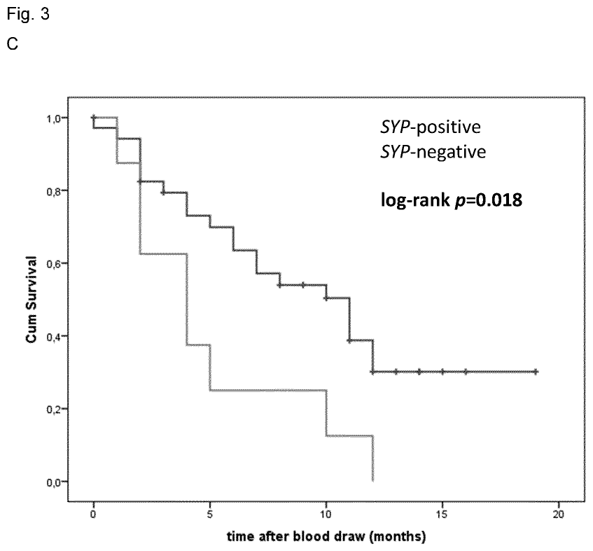

A negative impact of DLL3 and CgA on overall survival was observed (median OS 4 vs 9 months, log-rand p=0.035 and p=0.024), but not of EpCAM, and CK19 (FIG. 3). Likewise, SYP turned out to have a negative impact in those samples which had a lower amount of residual white blood cells (FIG. 3C).

### Conclusions

CTCs can be detected by a combined approach using the microfluidic Parsortix™ system and qPCR-based detection of CTC-related gene transcripts. Here the applicability of the approach was demonstrated for blood samples taken from lung cancer patients diagnosed with SCLC. The neuroendocrine markers CgA and SYP are detected in CTC-enriched blood fraction as a prognostic marker, which is of considerable interest for patient management regarding diagnoses and therapy monitoring. In addition, DLL3 was found to differentiate between NSCLC and SCLC, which can be used as a marker for monitoring NSCLC disease progression and for determining the NSCLC or SCLC patient's prognosis and therapy.

### Example 2

Patients and Methods

59 lung cancer patients with a histopathological confirmed diagnosis of SCLC were included into the study. A total of 65 blood samples were taken (n=33 at primary diagnosis; n=32 at disease progression). In addition, blood samples were taken from 19 healthy normal donors (HND) as control.

All blood samples were processed using the Parsortix™ system (Angle plc., UK) to increase the relative abundance of CTCs in the sample.

After harvesting and lysis of the captured cells, the total RNA was extracted and reverse transcribed into cDNA. Finally, qPCR was performed using TagMan® assays specific for CgA, SYP, and DLL3.

Results

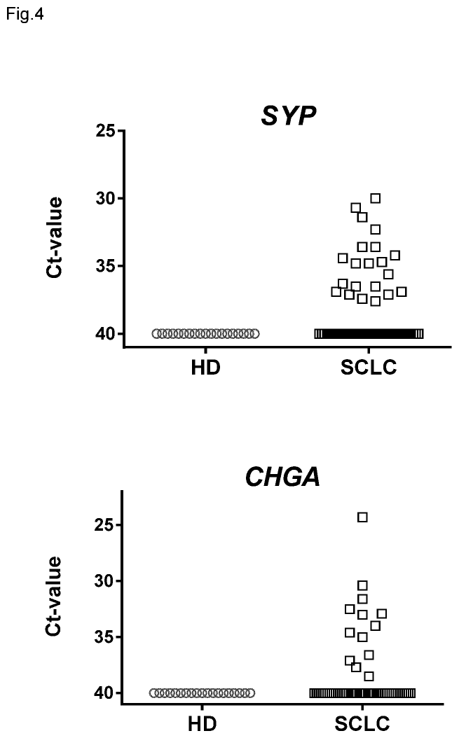

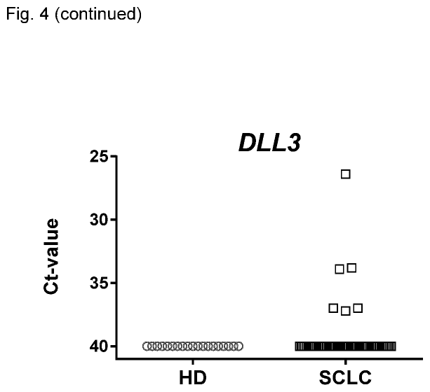

CgA, SYP and DLL3 transcripts were observed in 20.9%, 32.3% and 9.2% of the 65 SCLC samples, and in none of the 19 HND samples (FIG. 4).

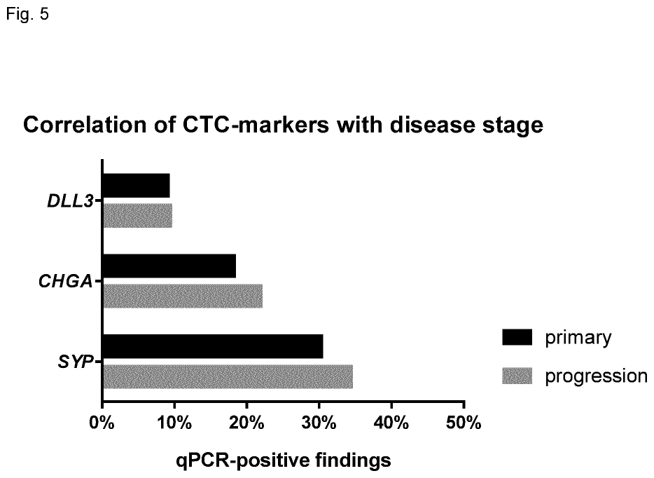

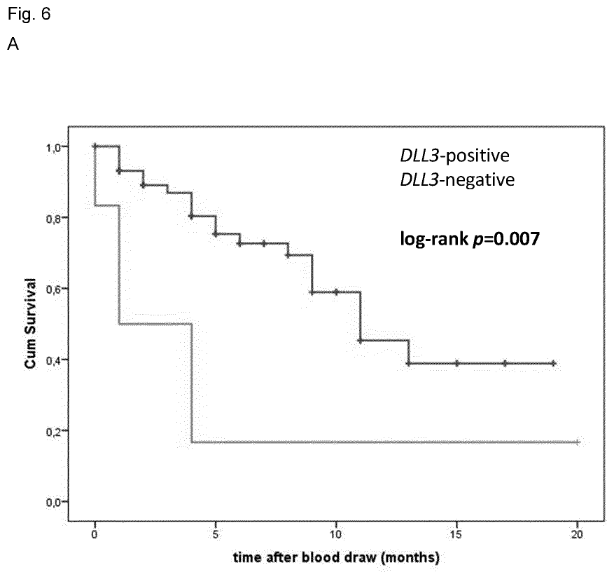

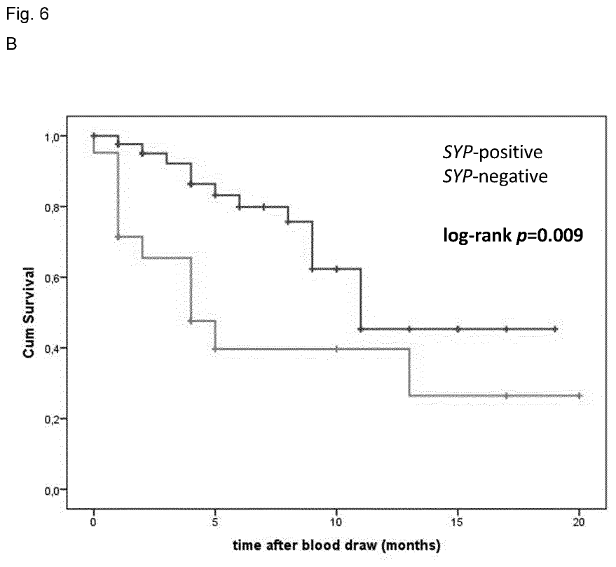

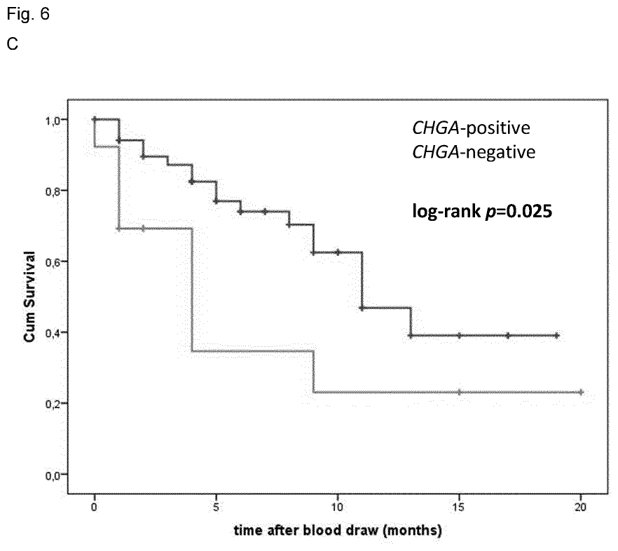

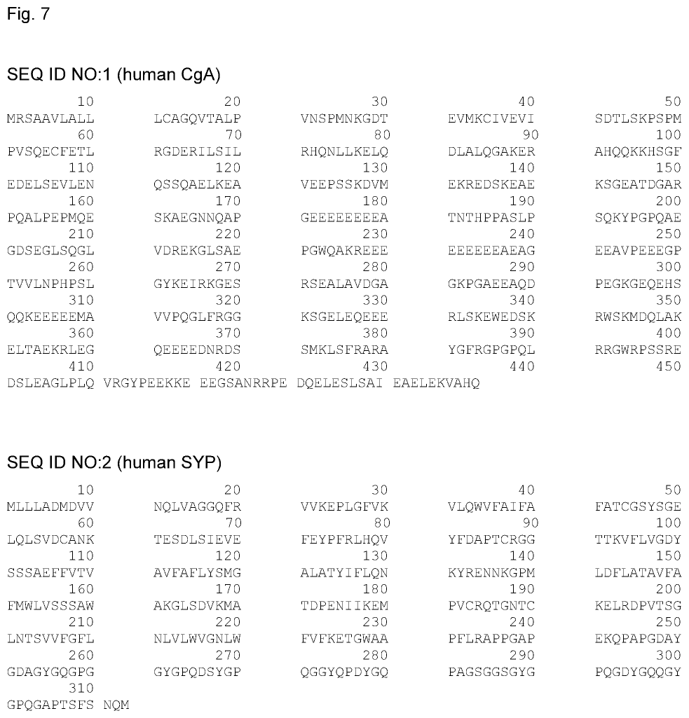

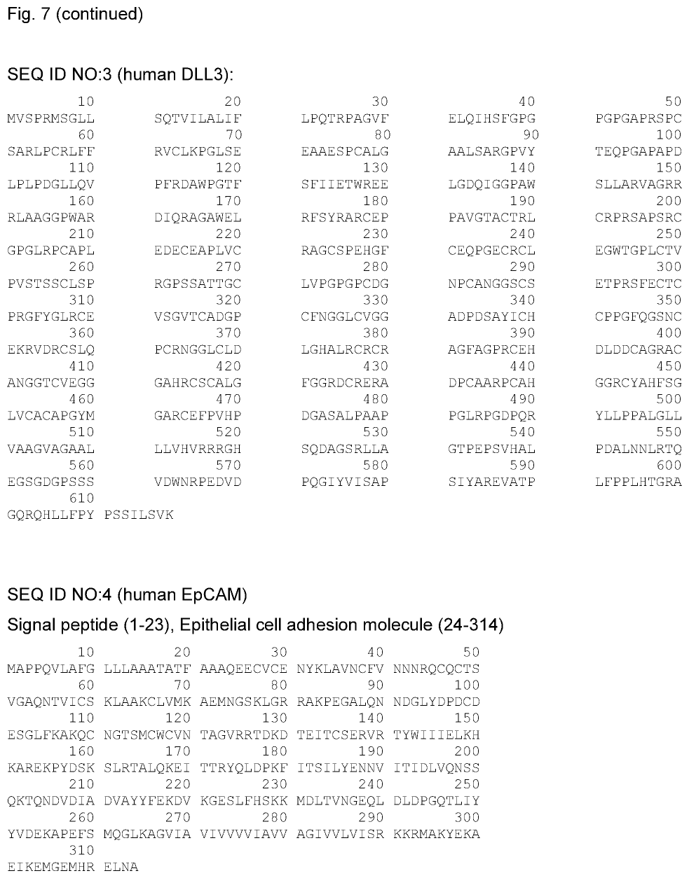

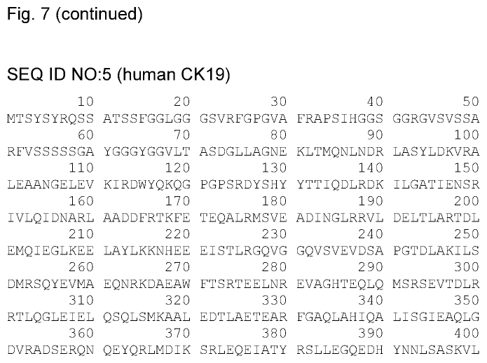

Overall, 26/65 (40.0%) samples were assigned as CTC-positive due to the expression of at least one marker out of CgA, DLL3 and SYP. The percentage of positive samples was not associated with the stage of disease when the blood sample was taken (FIG. 5).

The negative impact of CgA, SYP and DLL3 transcripts on overall survival as shown in Example 1 was confirmed.

1. A method of treating a patient with small-cell lung cancer (SCLC) who is at risk of disease progression, comprising:
identifying the SCLC patient at risk of disease progression by determining the expression of at least one circulating tumor cell marker in a blood sample or blood sample fraction of said patient, wherein said at least one marker is:
i) CgA and/or SYP; or
ii) DLL3 and at least one of CgA or SYP,
wherein expression of said at least one marker is indicative of the patient's prognosis of being at risk of disease progression; and
b) treating the SCLC patient for SCLC by administering a therapy used for treating advanced SCLC disease.

2. The method of claim 1, wherein said at least one marker is CgA and/or SYP.

3. The method of claim 1, wherein expression is determined by measuring the level of an expression product of said at least one marker.

4. The method of claim 1, wherein expression is determined by measuring RNA or protein expression.

5. The method of claim 1, wherein expression is determined by measuring an expression product by using RT-PCR or an immunoassay.

6. The method of claim 1, wherein an expression level of at least one marker is determined quantitatively or semi-quantitatively.

7. The method of claim 1, wherein said blood sample fraction is enriched in circulating tumor cells (CTCs).

8. The method of claim 1, wherein expression is determined by comparing the level of an expression product in said sample to a predetermined reference value, wherein an elevated level is indicative of the risk of an advanced SCLC disease condition and/or disease progression.

9. (canceled)

10. The method of claim 1, wherein an expression level of said at least one marker is determined at different time points during the therapy.

11. A method of monitoring treatment of an small-cell lung cancer (SCLC) patients with a SCLC therapy, comprising determining the expression of at least one marker of circulating tumor cells in a blood sample or blood sample fraction of said patient, wherein the marker is any one of:
i) CgA and/or SYP; or
ii) DLL3 and at least one of CgA or SYP,
wherein expression of said at least one marker is indicative of the patient's response to said therapy.

12. The method of claim 11, wherein blood samples are drawn from the SCLC patient at least twice, at two different time points during the therapy.

13-15. (canceled)

16. A diagnostic kit comprising a multimarker panel comprising one, two or three of the biomarkers CgA, SYP, and DLL3 and ligands specifically recognizing expression products of each marker of said multimarker panel.

17. The method of claim 1, wherein the therapy is selected from the group consisting of chemotherapy, radiotherapy, and immunotherapy.

18. The method of claim 17, wherein the radiation therapy is performed concurrently with chemotherapy.

19. The method of claim 17, wherein the radiation therapy is performed following a cycle of chemotherapy.

20. The method of claim 17, wherein the chemotherapy is performed with one or more drugs selected from the group consisting of carboplatin, etoposide, cisplatin, irinotecan, topotecan, and epirubicin.

21. The method of claim 1, wherein expression is determined using a diagnostic kit comprising, for each of said markers, a ligand specifically recognizing an expression product of said marker.

22. The method of claim 1, wherein the expression of at least two markers selected from the group consisting of DLL3, CgA, and SYP is determined.

23. The method of claim 11, wherein said expression is determined employing a diagnostic kit comprising, for each of said markers, a ligand specifically recognizing an expression product of said marker.

24. The method of claim 16, wherein the multimarker panel further comprises EpCAM and/or CK19.


SEQUENCE LISTING (5 sequences)

SEQ ID NO: 1 - protein, 450 aa, Homo sapiens
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQ

SEQ ID NO: 2 - protein, 313 aa, Homo sapiens
MLLLADMDVVNQLVAGGQFRVVKEPLGFVKVLQWVFAIFAFATCGSYSGELQLSVDCANKTESDLSIEVEFEYPFRLHQVYFDAPTCRGGTTKVFLVGDYSSSAEFFVTVAVFAFLYSMGALATYIFLQNKYRENNKGPMLDFLATAVFAFMWLVSSSAWAKGLSDVKMATDPENIIKEMPVCRQTGNTCKELRDPVTSGLNTSVVFGFLNLVLWVGNLWFVFKETGWAAPFLRAPPGAPEKQPAPGDAYGDAGYGQGPGGYGPQDSYGPQGGYQPDYGQPAGSGGSGYGPQGDYGQQGYGPQGAPTSFSNQM

SEQ ID NO: 3 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 4 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 5 - protein, 400 aa, Homo sapiens
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL